(19) United States
(12) Patent Application Publication
(10) Pub. No.: US 2022/0007983 A1
(43) Pub. Date: Jan. 13, 2022

(54) CONDUCTIVE POLYMERIC COMPOSITION

(71) Applicant: CONSCIOUS LABS SAS, LIMOGES (FR)

(72) Inventors: Michel ARMAND, Paris (FR); Shiyu ZOU, Strasbourg (FR); Julien DAUGUET, Paris (FR)

(21) Appl. No.: 17/413,081

(22) PCT Filed: Nov. 29, 2019
(86) PCT No.: PCT/FR2019/052866

§ 371 Date: Jul. 13, 2021

(30) Foreign Application Priority Data
Dec. 12, 2018 (FR) 1872804

(51) Int. Cl. A61B 5/268; C08F 16/06; C08K 13/02

(52) U.S. Cl. CPC A61B 5/268; C08F 16/06; C08K 5/053; C08F 2810/00; C08K 13/02

(57) ABSTRACT

The invention relates to an ionic conductive polymeric composition defined by the following general formula: (PH)x+(SOH)y+z(MCl); in which: —PH represents a polymer containing protic functions; —SOH represents a plasticizing polyol with a molecular mass of not less than 75 g/mol and not greater than 250 g/mol, in the form of discrete molecules; —MCl represents sodium or potassium chloride (M=Na or K); —0.3≤x/y≤3, x representing the amount by weight of the polymer PH, and y the amount by weight of the polyol SOH; —0.5%≤z≤15%, z representing the percentage by weight of MCl relative to the polyol SOH. Said polymeric composition may be used particularly as conductive material in electrodes for measuring electrophysiological signals.

The present invention relates to a polymer composition suitable for use as conductive material in electrodes for measuring electrophysiological signals.

The electrophysiological signals are the result of the electrochemical activity of living cells, which generates differences in electric potential, commonly referred to as “biopotentials”.

Measurement of the biopotential signals generated by the electrical activity of the cells is common practice in the medical field, for example in the context of electrocardiography (ECG) for studying heart function, or electroencephalography (EEG) for studying brain activity. For non-invasive exploration tests, this electrical activity is measured using electrodes positioned on the surface of the skin or scalp, at locations of the body that are chosen depending on the type of measurement to be taken.

Thus, for example, EEG consists in measuring the electrical activity of the brain by measuring the differences in electric potential between electrodes placed on the surface of the scalp.

The electrodes are used as transducers for converting the ionic current generated by the cell activity into electronic current.

Conventionally, electrodes used are usually constituted of a silver plate covered with a film of silver chloride (Ag/AgCl electrodes). These electrodes, which are used with a conductive aqueous gel applied between the skin and the electrode, are referred to as “wet electrodes”.

The use of conductive gel makes it possible to lower the skin-electrode impedance by hydrating the stratum corneum of the epidermis which facilitates the transduction of the ionic current into electronic current. Moreover, the gel also makes it possible to maintain a better contact between the skin and the electrode in the case of movements of the subject, which limits the disturbances that may result from this movement.

However, wet electrodes have various drawbacks which are well known. Firstly, prior to positioning the electrodes, it is conventionally necessary to prepare chosen areas of the scalp by shaving followed by light abrasion and cleaning with alcohol to thin the stratum corneum. These operations require time and the intervention of an external operator. Moreover, the abrasive products used for the preparation as well as the conductive gel leave residues on the scalp and the hair and may even, in certain cases, cause irritation in subjects with sensitive skin. Although suitable for use for EEG measurements carried out in hospital, at the doctor'≤office or in a research laboratory, they are not suitable for use in the field or in an EEG device that would be intended for the general public.

It has been proposed to use so-called “dry” electrodes, which have the advantage of not requiring the use of gel (for a review, see Lopez-Gordo, et al., Sensors 2014, 14(7), 12847-12870).

Most dry electrodes are metallic (mainly Ag/AgCl) and rigid and have conductive micro-spikes. The absence of gel is compensated for by the fact that these spikes can penetrate the stratum corneum and are thus able to directly pick up the ionic currents. These electrodes have a low impedance, but their rigidity and the presence of the spikes make them uncomfortable.

More recently, still to improve the comfort and ease of use of EEG devices intended in particular for the general public, electrodes that are nonmetallic, and even flexible, have appeared. In particular, electrodes made of polymer filled with particles of an electronically-conductive material have been produced. Although these electrodes are clearly more comfortable than the metallic electrodes and may be relatively good electronic conductors, they are very poor ionic conductors and have a much worse measurement performance compared to Ag/AgCl electrodes+electrolytic gel, or even compared to metallic rigid dry electrodes. One way of compensating for this very low ionic conductivity consists in making them active, i.e. adding a pre-amplification circuit to the source of the measurement. This makes it possible to artificially amplify the low current measured and therefore to reduce the resulting impedance making the measurement more robust and less sensitive to the noise inherent to the EEG. However, this solution only provides a small improvement, the electrode remains a very low ionic conductor. Furthermore, the addition of an active circuit increases the set-up cost and complexity (additional cables, increased rigidity of the wiring) and is notably not at all optimal in the case of a cap comprising a large number of sensors, or in the case of seeking a minimal cost (general public device). The possible replacement of the electrode is also more complex and more expensive.

The objective of the present invention is to provide dry electrodes comprising or constituted of a flexible polymer, and that do not have the drawbacks of the dry electrodes known in the prior art, in particular that do not require the addition of a pre-amplification circuit.

For this purpose, the present invention provides a polymer material specifically suitable for the manufacture of such electrodes.

One subject of the present invention is an ionically-conductive polymer composition defined by the following general formula:

(PH)x+(SOH)y+z(MCl);

wherein:
PH represents a polymer containing protic functions;
SOH represents a plasticizing polyol having a molecular mass greater than or equal to 75 g/mol and less than or equal to 250 g/mol, in the form of discrete molecules;
MCl represents sodium chloride or potassium chloride (M=Na or K);
0.3≤x/y≤3, x representing the amount by weight of the polymer PH, and y representing the amount by weight of the polyol SOH;
0.5%≤z≤15%, z representing the weight percentage of MCl relative to the polyol SOH.

This polymer composition is in the form of a flexible elastomer that is dry to the touch, and that does not exude the component SOH.

The expression “polymer containing protic functions” is understood here to mean a polymer, the chain of which contains functional groups capable of donating H+ ions to their surroundings. These may in particular be hydroxyl groups or amide groups.

The polymer PH may thus notably be a hydrolysis product of poly(vinyl acetate), having a degree of saponification of greater than or equal to 60% and less than or equal to 100%, preferably greater than or equal to 80% and less than or equal to 100%, and having an average molecular mass (M_{w}) of greater than or equal to 5×10⁴ and less than or equal to 2×10⁶ daltons, preferably greater than or equal to 1×10⁵ and less than or equal to 1×10⁶ daltons. Such polymers are known under the name of poly(vinyl alcohol)s (abbreviated hereinbelow to PVA).

Another example of a polymer PH is polyacrylamide (PAA) having an average molecular mass (M_{w}) of greater than or equal to 5×10⁴ and less than or equal to 5×10⁶ daltons.

The plasticizing polyol SOH may for example be chosen from glycerol, propylene glycol, dipropylene glycol or mixtures thereof. Preferably, SOH is glycerol or dipropylene glycol, and very preferably SOH is glycerol.

Preferentially:
the x/y ratio is such that 0.50≤x/y≤1.00, in particular 0.60≤x/y≤0.80, preferably 0.62≤x/y≤0.75, advantageously 0.63≤x/y≤0.71, and particularly preferably 0.64 s x/y≤0.69,
the percentage z is such that 1%≤z≤15%, advantageously 4%≤z≤6%, and preferably 4.5%≤z≤5.5%.

The ionic conductivity properties of the polymer composition in accordance with the invention make it particularly suitable for use in electrodes for measuring electrophysiological signals, in particular in electrodes intended for EEG.

It is possible to also give an ionically-conductive polymer composition in accordance with the invention a good electronic conductivity by adding thereto one or more electronically-conductive particulate carbon-based additive(s), and in particular, as nonlimiting examples, one or more carbon-based additive(s), such as graphites, graphite fibers, carbon black powders, and carbon fibers and nanotubes.

Consequently, according to one preferred embodiment of a polymer composition in accordance with the invention, it further contains an electrically-conductive particulate carbon-based filler.

Advantageously, the weight percentage of the conductive filler relative to the polymer PH is from 5% to 60%, preferably from 10% to 50%, advantageously from 20% to 50%.

To further improve the conductive properties of a polymer composition in accordance with the invention, it is also possible to add thereto a redox couple that enables the transition from ionic conductivity to electronic conductivity. Advantageously, the redox couple is an Ag/AgCl mixture, which may be added in the form of powder to the other constituents, in a proportion of from 1% to 8% by weight relative to the polymer PH.

Another subject of the present invention is:
an electrode for measuring an electrophysiological signal comprising a polymer composition in accordance with the invention;
a device for measuring an electrophysiological signal comprising one or more electrodes in accordance with the invention.

The present invention will be better understood with the aid of the remainder of the description which follows, which refers to nonlimiting examples describing the preparation and the properties of conductive polymer compositions and of electrodes in accordance with the invention.

### EXAMPLE 1: PREPARATION OF CONDUCTIVE POLYMER COMPOSITIONS

Mixtures in various proportions of polyvinyl alcohol, glycerol and sodium chloride were produced.

The polyvinyl alcohol (M_{w}˜195,000, SIGMA-ALDRICH), the glycerol (reagent grade, ≥99.0% (GC), SIGMA-ALDRICH) and the sodium chloride are weighed in a beaker and dissolved in demineralized water (PVA/water weight ratio=1:10) by heating to around 60° C. for around 1 hour, with stirring using a magnetic stirrer bar.

In another series of experiments, a polymer composition filled with graphite powder was prepared, according to the protocol described above, except that the graphite powder is added to the other constituents prior to dissolving. Various concentrations of graphite powder (Graphit GNP 12, purity 99.5%, particle size 16-63 μm) were tested.

### EXAMPLE 2: MANUFACTURE OF ELECTRODES

When the solution of polymer composition has reached a viscosity sufficient to stop the rotation of the magnetic stirrer bar, it can be used for the manufacture of the electrodes.

Flat electrode: This electrode is prepared by immersing a Gold Cup (OpenBCI) passive gold electrode in the solution of polymer composition for a few moments. Once the gold electrode is coated with composition, the assembly is left to dry in the open air and at room temperature for at least 3 days approximately.

Spiked electrode: An electrode mold with spikes is manufactured by 3D printing (material: polylactic acid). This mold is filled with the solution of polymer composition and a Gold Cup electrode is then immersed therein. The assembly is left to dry in the open air and at room temperature for at least 1 week, before removing from the mold.

### EXAMPLE 3: TEST OF THE CONDUCTIVE PROPERTIES OF THE ELECTRODES

### 1) Measurement of the Signal-to-Noise Ratio

The signal-to-noise ratio (SNR) is a ratio of signal power to noise power. It is a measure of the fidelity of signal transmission.

In order to determine it, the electrodes manufactured as described above were tested to measure the α (8-12 Hz) activity by EEG.

### EEG Setup:

3 electrodes were used for each measurement: a measurement electrode, a reference electrode, and a polarization (bias) electrode.

In the case of the flat electrodes, all the electrodes were placed on areas of hairless skin, namely on the forehead for the measurement electrode, and on the lobe of each ear for the reference electrode and the bias electrode.

In the case of using spiked electrodes, the measurement electrode is placed on the top of the cranium (vertex: Cz position according to the International System 10-20) and the reference and bias electrodes on the lobe of each ear.

The measurements are carried out over 2 sessions, of 2 minutes each, 1 minute with eyes open, and 1 minute with eyes closed (the power in the α band increasing when the eyes are closed).

### Calculation of the Signal-to-Noise Ratio:

The relative power of alpha activity is calculated using the following formula: Relative power=alpha (8-12 Hz) power/Total power of the signal (1-60 Hz) The signal-to-noise ratio (SNR) is then calculated as described by Tautan et al. (Proc. 7th International Conference on Biomedical Electronics and Devices; Biodevices 2014).

The higher the SNR, the more sensitive the electrode.

### Influence of the PVA:Glycerol Ratio on the Signal-to-Noise Ratio

The SNR is calculated as described above, for various PVA:glycerol proportions, with a constant concentration of NaCl of 5% by weight relative to the glycerol. The amount of PVA is used as reference. The theoretical proportion of glycerol increases from 0.66 to 1.75.

The results are illustrated by table 1 below:

TABLE 1

| PVA:Glycerol | SNR | Average SNR |
|---|---|---|
| 1:0.66 | / | / |
| 1:1.03 | 4.1083 | 5.1026 |
| 1:1.01 | 5.3627 | |
| 1:1.01 | 6.0968 | |
| 1:1.55 | 5.3691 | 5.4716 |
| 1:1.52 | 4.9488 | |
| 1:1.53 | 6.0968 | |
| 1:1.75 | 5.2216 | / |

For the lowest amounts of glycerol (PVA:glycerol ratio<1), no EEG signal was able to be detected. By increasing the proportion of glycerol, the SNR increases, but decreases again for the highest amount of glycerol. Furthermore, in the case of the PVA:glycerol ratio of 1:1.75 glycerol, exudation of glycerol after drying is observed, making the electrode unsuitable for use.

### Influence of the Concentration of NaCl on the Signal-to-Noise Ratio

The influence of the concentration of NaCl was then tested. The results are illustrated in table 2 below. The % of NaCl indicated are weight percentages relative to the glycerol (the saturation concentration of NaCl in the glycerol is 7.5%).

TABLE 2

| PVA:Glycerol | NaCl (g) | [NaCl] (% w/w) | SNR | Average SNR |
|---|---|---|---|---|
| 1:1.52 | 0.04 | 3.15 | / | / |
| 1:1.55 | 0.05 | 4.90 | 5.3691 | 5.4716 |
| 1:1.52 | 0.10 | 5.00 | 4.9488 | |
| 1:1.53 | 0.08 | 4.95 | 6.0968 | |
| 1:1.46 | 0.08 | 6.84 | 4.0919 | 3.6745 |
| 1:1.51 | 0.08 | 6.96 | 3.4859 | |
| 1:1.46 | 0.07 | 6.86 | 3.4457 | |
| 1:1.52 | 0.12 | 9.76 | 6.4631 | 6.1944 |
| 1:1.48 | 0.10 | 9.80 | 5.8284 | |
| 1:1.47 | 0.11 | 11.00 | 6.2917 | |

For the lowest concentrations of NaCl, no EEG signal was able to be detected. When the concentration increases, the signal becomes detectable and the SNR increases to reach an optimum at around 5% NaCl. However, when the saturation concentration is approached, the SNR decreases. It is assumed that this decrease could be due to the fact that the presence of too large an amount of ions hinders the mobility thereof. The SNR increases again for supersaturated concentrations. This could be explained by the deposition of salts on the surface of the electrodes after drying, which would increase the conductivity. However, at these high concentrations of NaCl, a deterioration of the surface of the electrodes, which take on an oily appearance and are unsuitable for use, is also observed.

Tests were also carried out with polymer compounds filled with graphite powder, to evaluate the influence of the graphite filler.

The results are illustrated in table 3 below. As above, the % of NaCl indicated are weight percentages relative to the glycerol.

TABLE 3

| Graphite (g) | PVA:Glycerol:Graphite | [NaCl] (% w/w) | SNR |
|---|---|---|---|
| 0.12 | 1:1.52:0.18 | 5.83 | / |
| 1.01 | 1:1.50:0.51 | 5.67 | 8.5763 |

For the lowest amount of graphite (18% by weight of PVA) no signal is detected. However, for a larger amount, a significant increase in the SNR is observed.

### 2) Measurement of the Ionic Conductivity

The ionic conductivity properties of an electrode of the invention (PVA:glycerol ratio=1:1.52; % by weight of NaCl relative to the glycerol=5%) were compared with those of dry electrodes from the prior art: FOCUS Dry Active EEG Electrodes (TRANSCRANIAL); Flex Sensor (COGNIONICS); DREEM electrode (DREEM).

The impedance of each of the electrodes was measured using an Analog Discovery 2 (DIGILENT) multimeter, and the results represented in the form of a Nyquist diagram.

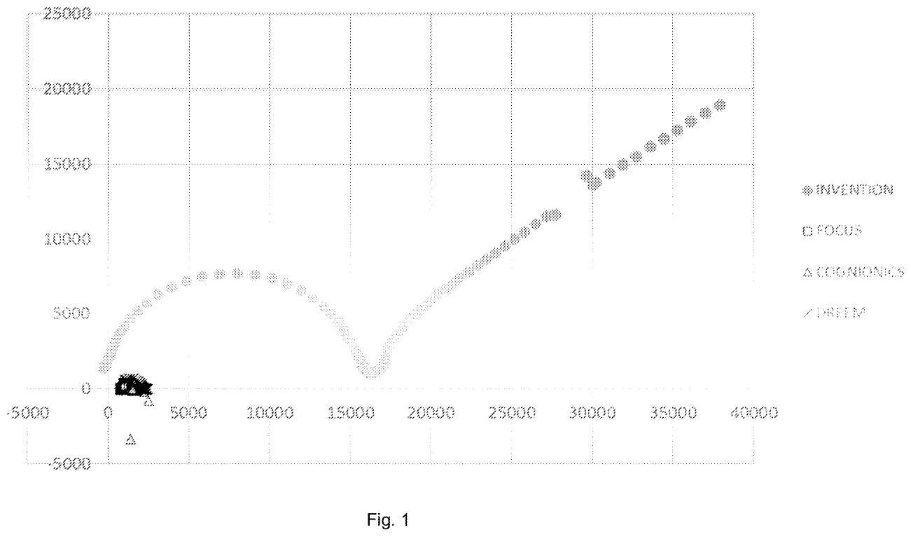

The results are illustrated by FIG. 1.

Key to FIG. 1: x-axis: real part of impedance Z′ (in ohms); y-axis: imaginary part of impedance Z″ (in ohms); [symbol] : electrode of the invention; Δ: COGNIONICS electrode; X: DREEM electrode; □: FOCUS electrode.

In the case of the electrode of the invention, the plot of the diagram is formed by a semicircle and a straight line. The semicircle represents a relaxation due to the movement of the ions at high frequencies and the straight line at low frequency represents the polarization at the electrodes. This plot confirms that this electrode is an ionic conductor.

In the case of the electrodes from the prior art, the plot of the diagram mainly shows clusters of points grouped on the x-axis, and no semicircle representing the movement of the ions is observed. This indicates that the materials of these electrodes are electronic conductors but are not ionic conductors.

1. An ionically-conductive polymer composition defined by the following general formula:
(PH)x+(SOH)y+z(MCl);
wherein:
PH represents a polymer containing protic functions constituted by hydroxyl groups;
SOH represents a plasticizing polyol having a molecular mass greater than or equal to 75 g/mol and less than or equal to 250 g/mol, in the form of discrete molecules;
MCl represents sodium chloride or potassium chloride (M=Na or K);
0.3≤x/y≤3, x representing the amount by weight of the polymer PH, and y representing the amount by weight of the polyol SOH;
0.5%≤z≤15%, z representing the weight percentage of MCl relative to the polyol SOH.

2. The polymer composition according to claim 1, wherein the polymer PH is a poly(vinyl alcohol) with a degree of saponification of greater than or equal to 60% and less than or equal to 100%, and an average molecular mass M_{w} of greater than or equal to 5×10⁴.

3. The polymer composition according to claim 1, wherein the plasticizing polyol SOH is chosen from glycerol, propylene glycol, dipropylene glycol or mixtures thereof.

4. The polymer composition according to claim 1, wherein the ratio x/y is such that 0.50≤x/y≤1.00.

5. The polymer composition according to claim 1, wherein the percentage z is 1%≤z≤15%.

6. The polymer composition according to claim 1, wherein it further comprises an electrically-conductive particulate carbon-based filler, and in that the weight percentage of said conductive filler relative to the polymer PH is from 20% to 50%.

7. The polymer composition according to claim 6, further comprising a redox couple enabling the transition from ionic conductivity to electronic conductivity.

8. An electrode for measuring an electrophysiological signal comprising a polymer composition according to claim 1.

9. A device for measuring an electrophysiological signal comprising one or more electrodes according to claim 8.
